(19) United States
(12) Patent Application Publication
(10) Pub. No.: US 2024/0016769 A1
(43) Pub. Date: Jan. 18, 2024

(54) Method for preparing compound or biological drug enhancing CNPase activity for treating heart diseases

(71) Applicant: ZHUHAI YUANZHI HEALTH TECH CO. LTD., Guangdong (CN)

(72) Inventors: Dongfang Wang, (US); Kaixin CHEN, (US); Zhiqiang LU, (US); Wen TAN, (US)

(21) Appl. No.: 18/036,404

(22) PCT Filed: Nov. 14, 2021
(86) PCT No.: PCT/CN2021/130496

§ 371 Date: May 11, 2023

(30) Foreign Application Priority Data
Nov. 15, 2020 (CN) 202011274161.1

(51) Int. Cl. A61K 31/19; C12N 15/113; A61K 48/00; A61P 9/04

(52) U.S. Cl. CPC A61K 31/19; C12N 15/1137; A61K 48/0041; A61P 9/04; C12N 2310/141

(57) ABSTRACT

A method for preparing compound or biological drug enhancing CNPase activity for treating heart diseases. The chemical method relates to using kaurane compounds to up-regulating the expression and activity of CNPase. Biological therapeutic drugs related to the preparation of CNPase enzyme expressed by using recombinant adeno-associated virus and local interventional therapy. In a rat myocardial hypertrophy and heart failure model, the methods can effectively improve myocardial hypertrophy, myocardial remodeling, inhibit myocardial hypertrophy and fibrosis, and increase cardiac function.

### BACKGROUND OF INVENTION

2′, 3′-cyclic nucleotide-3′-phosphodiesterase (CNPase) was found in the early 1960s. It has the function of catalyzing the degradation of 2′, 3′-CAMP and 2′, 3′-cGMP and is highly expressed in the central nervous system. The release of extracellular 2′, 3′-CAMP is related to injury, and 2′, 3′-CAMP can activate mitochondrial permeability transition pores (mPTPs) and lead to apoptosis. At present, it is found that 2′, 3′-CAMP, and CNPase are related to mitochondrial membrane permeability. Jackson EK et al. reported that CNPase knockout could protect renal function after ischemia/reperfusion (J am SOC Nephrol. 2016). So far, no one has reported the target and function of CNPase in cardiomyopathy.

Heart failure is a severe manifestation or late stage of various heart diseases, the mortality and readmission rate remain high. Heart failure is a complex clinical syndrome caused by a variety of causes of abnormal changes in cardiac structure and/or function, resulting in dysfunction of ventricular contraction and/or diastole. It mainly manifests as dyspnea, fatigue, and fluid retention (pulmonary congestion, circulation congestion, and peripheral edema). Cardiac remodeling refers to changes in the size, shape, and function of the heart due to changes in molecular and gene expression during cardiac injury or hemodynamic stress response.

Left ventricular hypertrophy is an adaptive response of the heart to pathological conditions such as pressure or volume overload, sarcomere protein gene mutation, or myocardial infarction, which is accompanied by a variety of heart diseases, such as hypertension, valve disease, and ischemic heart disease. In the pathological process of these diseases, excessive pressure load will lead to concentric myocardial hypertrophy, which is considered a compensatory pathological change because it can increase left ventricular contractility and reduce ventricular wall pressure and myocardial oxygen consumption. However, left ventricular hypertrophy can also be a risk factor for severe heart failure and malignant arrhythmia. Therefore, it is of essential clinical significance to effectively inhibit left ventricular myocardial hypertrophy without causing circulatory dysfunction.

Among the many adverse factors caused by myocardial hypertrophy, abnormal energy metabolism is particularly prominent. Under the condition of left ventricular myocardial hypertrophy, the model of cardiac energy metabolism mainly depends on aerobic oxidation of long-chain fatty acids to aerobic glucose oxidation. On the one hand, the change of energy metabolism substrate can reduce the oxygen consumption required to produce each mole of ATP. That is, it can reduce the production of reactive oxygen species (ROS) in cells; On the other hand, this change inevitably has a large number of adverse factors, such as chronic fatty acid aerobic oxidation disorder, lactic acid accumulation and increased glycolysis in cardiomyocytes which leads to the decrease of total ATP level.

Our team used the rat model of pressure-induced chronic myocardial hypertrophy constructed by abdominal aortic ligation and found that the expression of CNPase protein did not change. Still, the enzyme activity decreased, suggesting that the insufficient activity of CNPase may cause myocardial hypertrophy. Our team directly took CNPase as the target, and overexpression of CNPase in the heart can reverse the course of myocardial hypertrophy. It is proved that overexpression of CNPase can reverse myocardial hypertrophy and compensatory cardiac function injury in the process of chronic ischemia.

The applicant of this patent disclosed the sub localization of CNPase in mitochondria through immunofluorescence technology and disclosed explored its function of affecting mitochondrial energy supply; The recombinant AAV packaging plasmid expressing CNPase protein was constructed by molecular cloning technology. The recombinant AAV2/9-cnpase virus was prepared by virus packaging and purification technology. This patent disclosed the protective function and clinical therapeutic potential of overexpression of CNPase on cardiomyopathy determined by the model of compensatory cardiac function in myocardial hypertrophy and chronic ischemia in rats. At the same time, the invention discloses that kaurane compounds, such as sodium isosteviol which enhance the enzyme activity of CNPase.

### DETAILS OF INVENTION

The invention aims to provide a new tool for enhancing the activity or expression of CNPase in the heart, including the use of biological drugs of adeno-associated virus and compound sodium isosteviol, which can enhance the activity of CNPase, and protective drugs for heart failure and myocardial remodeling. The patients in the preclinical heart failure stage have no symptoms or signs of heart failure. Still, they develop into structural heart diseases, such as left ventricular hypertrophy, asymptomatic valvular heart disease, and so on. The invention discloses that kaurane compounds, such as isosteviol and its derivatives, can enhance the activity of CNPase for the treatment of heart failure and myocardial remodeling.

The biotherapy method of the invention includes recombinant vector construction, recombinant virus packaging, concentration, purification, and corresponding identification. Specifically, the AAV-CNPase vector was constructed by AAV overexpression vector and molecular cloning technology. Its expression activity was identified by transient transfection and Western blot. Further, the recombinant AAV2/9-CNPase virus is based on the steps of virus coating, virus particle collection, concentration and purification, titer identification, and so on. The recombinant AAV2/9-CNPase virus of the invention is used to prevent and/or treat diseases related to compensatory cardiac function injury in the process of myocardial hypertrophy and chronic ischemia. Further, the AAV2/9-CNPase can realize the up-regulation of myocardial-specific CNPase and to prevent and/or treat the diseases related to compensatory cardiac function injury in the process of myocardial hypertrophy and chronic ischemia in rats.

Specifically, the chronic myocardial hypertrophy disease model of rats was established by abdominal aortic ligation. Each rat was injected 1×10¹² AAV9-CNPase under ultrasound guidance. Rats were treated in the second week, and the hearts were harvested. Fluorescence quantitative PCR and Western blot were used to confirm that the targeted injection of recombinant AAV2/9-CNPasee can improve the expression of

CNPase mRNA and protein. Further, ultrasound was used in the second, fourth, sixth, and eighth weeks after virus injection. The detection parameters include ejection fraction, shortening fraction, diastolic blood pressure, systolic blood pressure, LVSP, max pressure, Max dp/dt, and other kinetic parameters.

Further, in the eighth week after the virus injection, the rats were scarified. The hearts were collected, fixed with electron microscope fixing solution, and detected by scanning electron microscope after the preparation of electron microscope film. The observation of mitochondrial morphology confirmed that CNPase injection can promote mitochondrial autophagy and alter the changes of cardiac energy homeostasis by promoting the clearance of damaged mitochondria. At the eighth week after virus injection, the rats were scarified, the hearts were collected, the total protein was extracted. CNPase can inhibit the activation of TGF-β1/2 signal pathway according western blot.

Specifically, the myocardial ischemia model of rats was established by coronary artery ligation. Each rat was injected with 1×10¹² AAV-CNPase under ultrasound guidance. Ejection fraction, shortening fraction, and other cardiac function indexes were examined at fourth week. Specifically, the use of CNPase enzyme as protective drugs for myocardial ischemic injury-related diseases. Myocardial ischemic injury-related diseases are heart failure, arrhythmia, ischemic cardiomyopathy, and cardiac rupture. The invention reveals the direct relationship between CNPase and cardiac function for the first time, demonstrating that CNPase plays a protective role in the compensatory cardiac function in myocardial hypertrophy and chronic ischemia. Up-regulation of CNPase can prevent/protect the myocardial hypertrophy and myocardial diseases. It also provides a new scheme for the prevention and treatment of heart failure, arrhythmia, ischemic cardiomyopathy, heart rupture and other diseases related to myocardial hypertrophy, myocardial fibrosis, and myocardial ischemic injury, and has a broad clinical application prospect.

The above is a general introduction to the invention. To better illustrate the method and technology of the invention, an embodiment case will be given below so that it can be performed by those skilled in the art.

The methods and embodiment of the present invention are provided in detail in the following embodiment.

### EXAMPLES

To further explain the technology used to achieve the purpose of the invention, detailed methods, technologies, processes, and characteristics for determining and identifying the pharmaceutical and therapeutic uses of the compounds in the invention are described below. The case provides experimental methods and results for supporting and verifying the animal model used in the invention. The cases involved used appropriate control group experiments and statistical analysis methods. The following examples are used to describe rather than limit the application of the invention. The methods and techniques involved in these cases can prepare chemical or biological drugs that enhance CNPase activity. Same method can be used to evaluate the therapeutic effect of other compound preparations.

The cases enumerated in the invention are used to support the experimental method and results and verify the animal model used in the invention. All experiments of the invention adopt appropriate control and statistical tests. The following embodiments are provided to illustrate rather than limit the present invention. These examples illustrate a method for preparing chemical or biological drugs that enhance CNPase activity for the treatment of heart failure and myocardial remodeling.

### Experimental Materials

Experimental animals: adult male Sprague Dawley rats, weighing 230 g±20 g, aged 6-8 weeks. The feeding environment includes constant temperature, humidity, and a strict dark-light cycle.
Construction of AAV9-CNPase recombinant plasmid: SD rats were killed by cervical dislocation method, the heart was harvested, the appropriate weight of heart tissue was weighed, and the total RNA was extracted by Trizol method; Take five μ G RNA was used as a template, and cDNA was prepared by RNA reverse transcription kit of Qiagen company; Using cDNA as template and Thermo Scientific™ Phusion™ CNPase coding sequence (CDS) was amplified by high fidelity DNA polymerase, in which the primers were BamHI and HindIII with enzyme digestion sites; The PCR products were separated and purified by agarose electrophoresis, digested and purified by BamHI and HindIII to prepare CNPase CDs products with viscous ends of enzyme digestion sites. PAAV MCS plasmids were digested and purified by BamHI and HindIII to prepare linear plasmids; The plasmid and CNPase CDs products were digested by T4 ligase and transferred into E. coli competent DH5α cells were incubated in LB medium at 37° ° C.for one hour; After centrifugation, take the precipitate, resuspend it, evenly apply it to the agarose solid plate with ampicillin antibiotics, and culture it at 37° C.overnight; Monoclonal antibodies were extracted, positive clones were screened by colony PCR, and the construction of recombinant plasmid was confirmed by sequencing.

Packaging, purification, concentration and titer identification of recombinant AAV9-CNPase: HEK293 cell line is used as a tool for packaging recombinant virus. The specific steps of virus packaging are as follows: pAAV MCS CNPase plasmid, phelper plasmid and pAAV-rc9 plasmid without endotoxin are extracted by Plasmid Extraction Kit, and transfected by liposome transfection; Collect the culture medium supernatant and cells respectively, precipitate and concentrate the supernatant with PEG6000, obtain the virus particles in the cells by repeated freezing and thawing, and concentrate with PEG6000; The concentrated virus was purified by ultra-high-speed centrifugation and dialysis, sub packed and frozen at −80° C.for standby; The virus titer was detected by quantitative fluorescence PCR.

### Experimental Method

Anesthesia was performed by intraperitoneal injection of 10% chloral hydrate 0.3 ml/100 g. After anesthesia, the rats were supine and their limbs were fixed. Do abdominal skin preparation. The operation site was located along the middle of the lower abdomen of the xiphoid process and above the left kidney. A longitudinal incision of 2.0˜2.5 cm was made, and the abdominal cavity was opened layer by layer. Expose the posterior abdominal wall and left kidney, find the branches of the left and right renal arteries and blunt free the abdominal aorta above the left renal artery. Insert No. 1-0 surgical silk thread under the abdominal aortic segment between the branches of the left and right renal arteries, and place a blunt No. 7 syringe needle along the direction of the blood vessel. The abdominal aorta and nylon suture were ligated between the branches of the left and right renal arteries. Observe the whitening of the left kidney (indicating that the ligation is reliable), then quickly draw out the syringe needle, observe the congestion and redness of the left kidney, and reduce the cross-sectional area of the abdominal aorta about 50%. Suture layer by layer after the operation. Gentamicin was injected intraperitoneally for 3 days to prevent infection. After laparotomy, the silk thread was threaded through the abdominal aorta in the sham operation group. Except that the abdominal aorta was not narrowed, other operations were the same as those in the formal operation group. At the end of the observation period, after measuring the hemodynamics in vivo, all animals were scarified, and the heart was harvested for further analysis.

### Measurement of Cardiac Hemodynamic Parameters

In the second week after modeling, and in the sixth week after AAV9-CNPase was given, the rats were anesthetized. The ECG needle electrode was inserted subcutaneously into the limbs (20% urethane, 12 g/kg, intraperitoneal injection). After the ECG and body temperature (37° C.) were stable, the right common carotid artery was separated, and the end of the common carotid artery was ligated for arterial intubation. The pressure sensor is connected with the multi-channel physiological signal acquisition and processing system. Systolic blood pressure (SBP), diastolic blood pressure (DBP), and heart rate (BPM) were recorded 15 minutes after stabilization. Then, the cannula was inserted into the left ventricle until the ventricular pressure waveform, left ventricular systolic pressure (LVSP), and left ventricular end-diastolic pressure (LVEDP) appeared. All (−dP/dtmax) were recorded 15 minutes after stabilization. All variables were monitored by power lab software (Power Lab 8/30 ad instruments, Australia).

### Histological Analysis

Rat myocardial tissue was fixed with 4% neutral formalin, embedded in paraffin, cut into 3mm sections, and then stained with hematoxylin-eosin (H&E) and Masson. Zeiss confocal microscope was used to take photos. H&E staining detected the morphology and size of cells were detected by H&E staining, and fibrosis was detected by Masson staining. Computer-aided image analysis (image processing software) determined the cross-sectional cell area and interstitial collagen content. The sample size is at least four or five different heart tissues.

### Statistical Analysis

The differences between multiple groups were compared by analysis of variance (one-way analysis of variance) and post-hoc Tukey's test. The P values of all tests were two-tailed, and P<0.05 was considered statistically significant.

### Example 1

This case mainly shows that sodium isosteviol enhances CNPase activity in angiotensin (Ang)-II induced cardiac hypertrophy cells. The enzyme activity of CNPase in hypertrophic myocardium was detected by the detection method of calf intestinal alkaline phosphatase activity.

TABLE 1
Sodium isosteviol can enhance CNPase activity in
cardiac hypertrophy induced by Ang-II (n = 3)

| | Control | Ang-II | STVNa + Ang II | STVNa |
|---|---|---|---|---|
| CNPase activity | 1.000 ± 0.2154 | 0.2266 ± 0.02390 | 0.9629 ± 0.411 | 0.9326 ± 0.241 |

### Example 2

This is mainly illustrating the effect of overexpression of CNPase on TAAC-induced myocardial hypertrophy.

Adult SD rats were treated with AAV9-CNPase after TAAC induction for 2 weeks. Heart weight ratio (HW/BW) is an index reflecting myocardial hypertrophy. In the 8-week TAAC model group, we found that the heart weight ratio (HW/BW) of rats in TAAC group increased significantly. Overexpression of CNPase effectively reduce the HW/BW ratio.

TABLE 1
Effects of overexpression of CNPase on heart weight
and body weight of TAAC model rats (n = 8-16)

| | Sham + | Sham + | TAAC + | TAAC + |
|---|---|---|---|---|
| | AAV9-GFP | AAV9-CNPase | AAV9-GFP | AAV9-CNPase |
| Heart weight (mg) | 1219 ± 71.49 | 1072 ± 80.99 | 1331 ± 156.1 | 1240 ± 162.1 |
| Body weight(g) | 431.5 ± 27.32 | 392.4 ± 27.70 | 397.6 ± 34.03 | 418.5 ± 33.25 |
| HW/BW (mg/g) | 2.819 ± 0.1488 | 2.733 ± 0.07610 | 3.335 ± 0.3126 | 2.957 ± 0.3201 |

### Example 3

This case mainly illustrates the role of AAV9-CNPase in inhibiting the formation of myocardial fibrosis.

To determine whether overexpression of CNPase can attenuate TAAC-induced myocardial fibrosis, we used Masson staining to detect the changes of left ventricular myocardial interstitial collagen. We found that myocardial fibrosis and collagen deposition increased in TAAC group. Overexpression of CNPase can effectively reduce myocardial fibrosis and collagen deposition.

TABLE 2
Overexpression of CNPase inhibits the formation of
myocardial fibrosis in TAAC model rats (n = 8-16)

| | Sham + | Sham + | TAAC + | TAAC + |
|---|---|---|---|---|
| | AAV9-GFP | AAV9-CNPase | AAV9-GFP | AAV9-CNPase |
| % Interstitial | 4.811 ± 4.517 | 0.3680 ± 0.2682 | 11.30 ± 5.680 | 3.610 ± 4.991 |
| fibrosis | | | | |

### Example 4

In order to evaluate whether CNPase can also improve the cardiac function of TAAC rats, we found that in the rat model of myocardial hypertrophy induced by TAAC, the heartbeat (BPM), left ventricular ejection fraction (EF), fractional shortening (FS) and cardiac output (CO) of CNPase overexpressed rats were improved.

TABLE 3
Cardiac specific overexpression of CNPase can effectively improve
cardiac function and other indexes of taac rats (n = 8-16)

| | Sham + | Sham + | TAAC + | TAAC + |
|---|---|---|---|---|
| | AAV9-GFP | AAV9-CNPase | AAV9-GFP | AAV9-CNPase |
| BPM | 344.1 ± 21.19 | 317.5 ± 41.91 | 333.9 ± 40.63 | 333.6 ± 32.43 |
| FS | 49.75 ± 3.411 | 47.92 ± 6.641 | 35.01 ± 6.564 | 45.73 ± 5.793 |
| EF | 79.48 ± 3.464 | 77.19 ± 6.727 | 62.03 ± 8.596 | 75.03 ± 6.536 |
| CO | 241.2 ± 14.89 | 255.6 ± 34.12 | 191.2 ± 35.44 | 236.2 ± 23.49 |

1. A method of increasing the activity or expression of myocardial CNPase, by administering kaurane compounds or by using biological methods for treatment of heart diseases.

2. The method of claim 1, wherein the said heart disease including myocardial ischemia, myocardial hypertrophy, heart failure, and myocardial remodeling.

3. The method of claim 1, wherein the said kaurane compounds representing structural formula (I), wherein R1: hydrogen, hydroxyl or alkoxy; R2: carboxyl group, carboxylate, acyl halide, aldehyde group, hydroxymethyl group, and ester group, acrylamide group, acyl group or ether bond group that can form carboxyl group; R3, R4, R5, R6, R8: oxygen, hydroxyl, hydroxymethyl, and ester or alkoxy methyl groups that can hydrolyze to hydroxymethyl; R7: methyl, hydroxyl, and ester group or alkoxy methyl that can hydrolyze to hydroxymethyl; R9: methylene or oxygen.

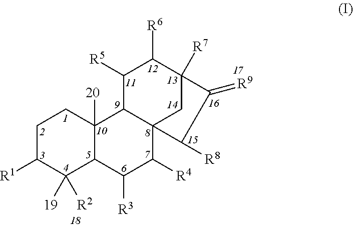

4. The method of claim 3, wherein the said kaurene comprising isosteviol, steviol, and their derivatives.

5. The method of claim 1, wherein the said biological method is recombinant gene method by utilizing lentivirus, adenovirus, and adeno-associated virus to increase the activity and expression of CNPase.

6. The method of claim 1, wherein the biological method is characterized by using non-coding RNA to increase the activity and expression of CNPase.

7. The method of claim 1, wherein the said increasing the activity or expression of myocardial CNPase, which is characterized by decreasing either the absolute amount or the concentration of related substrates including 2′, 3′-CAMP, 2′, 3′-CGMP, 2′, 3′-cCMP and 2′, 3′-cIMP.

8. The method of claim 1, wherein the said enhanced of CNPase activity, which is characterized by reduction of the ratio of the substrates including 2′, 3′-CAMP , 2′, 3′-cGMP, 2′, 3′-cCMP or 2′, 3′-cIMP related to the substrates either 3′, 5′-cGMP or 3′, 5 -CAMP in the tissue.

9. The method of claim 6, wherein the said non-coding RNA, is characterized by using a variety of RNA with known functions including rRNA, tRNA, snRNA, snoRNA and microRNA, and by using RNA with unknown functions in order to increase the activity of CNPase.

10. The method of claim 1, wherein the said increasing the activity or expression of myocardial CNPase by administering kaurane compounds or by using biological methods, which is characterized by intravenous, inhalation, or myocardium local injection of pharmaceutical excipient/carrier, which is also characterized by pharmaceutical formulation including injectable solution, injectable dry powder or other combined releasing agents.
